(19) United States
(12) Patent Application Publication
(10) Pub. No.: US 2025/0194898 A1
(43) Pub. Date: Jun. 19, 2025

(54) RELAY ADAPTER AND MEDICAL DEVICE SYSTEM

(71) Applicant: OLYMPUS MEDICAL SYSTEMS CORP., Tokyo (JP)

(72) Inventor: Takeo SUZUKI, Tokyo (JP)

(73) Assignee: OLYMPUS MEDICAL SYSTEMS CORP., Tokyo (JP)

(21) Appl. No.: 18/977,140

(22) Filed: Dec. 11, 2024

Related U.S. Application Data
(60) Provisional application No. 63/609,897, filed on Dec. 14, 2023.

(51) Int. Cl. A61B 1/00; A61B 1/06

(52) U.S. Cl. CPC A61B 1/00027; A61B 1/00097; A61B 1/00103; A61B 1/06

(57) ABSTRACT

A relay adapter includes a first connector extending in a first direction, the first connector configured to be connected to a first receptacle of a power supply device; and a main body including: a lateral body extending in a second direction intersecting the first direction; a second receptacle configured to be connected to a second connector of a medical device; and a grip provided at least one of (i) in an end surface of the lateral body in the second direction or (ii) between the second receptacle and the end surface.

### RELATED APPLICATION DATA

This application is based on and claims priority under 35 U.S.C. § 119 to U.S. Provisional Application No. 63/609,897, filed on Dec. 14, 2023, the entire contents of which are incorporated herein by reference.

### BACKGROUND

### 1. Field

The present disclosure relates to a relay adapter connecting a power supply device and a medical device, and a medical device system including the relay adapter.

### 2. Description of the Related Art

Conventionally, reusable endoscopes that are cleaned and disinfected and used multiple times are used as medical devices. The endoscope connects a connector of the endoscope to a receptacle such as an endoscope processor. The endoscope processor functions as a power supply devices to supply power to the endoscope. The endoscope processor performs image processing or the like on an image pickup signal received from the endoscope.

In contrast to the reusable endoscopes that can be used multiple times, single-use endoscopes, which are disposed of after a single use, have been proposed. The single-use endoscope may adopt different standards from the conventional reusable endoscope, such as using components with generic standards to reduce costs.

For the single-use endoscope with different standards from the conventional reusable endoscopes, an endoscope processor, a light source system, or the like, which are designed specifically for the single-use endoscope, are used, for example.

Meanwhile, there is demand to reduce the cost of purchasing new devices by using an endoscope processor, a light source system, or the like used for the conventional reusable endoscope for the single-use endoscope.

For example, especially a low-price single-use endoscope may differ from the reusable endoscope not only in terms of standards of signals and the like, but also in terms of hardware standards of connectors for connecting to the endoscope processor. The connectors of the low-price single-use endoscope has a simpler structure compared to a connector of the reusable endoscope.

To connect the single-use endoscope with different standards to the endoscope processor for the reusable endoscope, it is necessary to go through a relay adapter. Here, the single-use endoscopes is intended for single use, whereas the relay adapter is expected for multiple use.

For example, Japanese Patent Application Laid-Open Publication No. H05-337079 describes an adapter to enable endoscopes equipped with different −ypes of solid-state image pickup device to be connected to a control apparatus in a simplified manner.

In operations for multiple cases, single-use endoscopes are expected to be a greater number of cases per day than reusable endoscopes. In such operations, it is expected that the relay adapter is left inserted in the receptacle of the endoscope processor and only single-use endoscopes are attached or detached.

### SUMMARY

A relay adapter according to one aspect of the present disclosure includes a first connector extending in a first direction, the first connector configured to be connected to a first receptacle of a power supply device; and a main body including a lateral body extending in a second direction intersecting the first direction, a second receptacle configured to be connected to a second connector of a medical device, and a grip provided at least one of (i) in an end surface of the lateral body in the second direction or (ii) between the second receptacle and the end surface.

A medical device system according to one aspect of the present disclosure includes a medical device including a connector, a power supply device including a first receptacle, the power supply device configured to transmit power to be supplied to the medical device, and a relay adapter including a first connector extending in a first direction, the first connector configured to be connected to the first receptacle, and a main body. The main body includes a lateral body extending in a second direction intersecting the first direction, a second receptacle configured to be connected to the connector of the medical device, and a grip provided at least one of (i) in an end surface of the lateral body in the second direction or (ii) between the second receptacle and the end surface.

A relay adapter according to one aspect of the present disclosure includes a main body configured to be connected to a connector of a medical device, and a first connector configured to be connected to a first receptacle of a power supply device configured to power the medical device, the first connector extending in an axial direction from a middle portion of the main body. The main body and the first connector form a T-shape. The main body includes a lateral body extending in a direction intersecting the axial direction, a second receptacle configured to be connected to the connector of the medical device, and a grip provided at least one of (i) in an end surface of the lateral body in the second direction or (ii) between the second receptacle and the end surface.

### BRIEF DESCRIPTION OF THE DRAWINGS

FIG. 1 is a view showing a configuration of an endoscope system in a first embodiment of the present disclosure.

FIG. 2 is a partial exploded perspective view showing a configuration in which an endoscope and an endoscope processor are connected using a relay adapter in the above first embodiment.

FIG. 3 is a partial perspective view showing a state in which the endoscope and the endoscope processor are connected using the relay adapter in the above first embodiment.

FIG. 4 is a perspective view showing the relay adapter and a first connector of the endoscope in the above first embodiment.

FIG. 5 is a plan view showing a configuration of the relay adapter in the above first embodiment.

FIG. 6 is a partial enlarged perspective view showing a configuration when the relay adapter of the above first embodiment is connected to a first receptacle of the endoscope processor.

FIG. 7 is a block diagram showing an electrical configuration of the relay adapter in the above first embodiment.

### DETAILED DESCRIPTION

In general, if an endoscope chamber is narrow, for example, a user may have an impression that a protrusion of a relay adapter from an endoscope processor is large. Furthermore, a large protrusion of the relay adapter may restrict an operative space in the endoscope chamber.

Meanwhile, in order to avoid the relay adapter from protruding from the endoscope processor, it can be conceived that the relay adapter is operated by removing it from the endoscope processor each time after a single-use endoscope is used. However, this increases a workload on the user. If the number of cases when the single-use endoscopes are used is greater than the number of cases when conventional reusable endoscopes are used, the number of times the endoscope processor is attached to and detached from the receptacle of the endoscope processor is greater than before. This causes the receptacle of the endoscope processor to deteriorate faster than before.

If the relay adapter hardly protrudes from the endoscope processor, it may be difficult to attach or detach the relay adapter to or from the endoscope processor.

According to the embodiment described below, a relay adapter that capable of suppressing protrusion from a receptacle of an endoscope processor and easily attachable and detachable, and a medical device system including the relay adapter can be provided.

Hereinafter, an embodiment of the present disclosure will be described with reference to the drawings. However, the present disclosure is not limited by the embodiment described below.

Note that in the descriptions of the drawings, the same or corresponding component will be denoted by same reference signs as appropriate. Each drawings is schematically illustrated, and it should be noted that length relations of each elements, a ratio of lengths of each elements, the quantities of each elements, and the like may differ from real ones for the sake of brevity of the description in one drawing. Furthermore, even among a plurality of drawings, some parts may contain different length relations, ratios, quantities, or the like to each other.

### First Embodiment

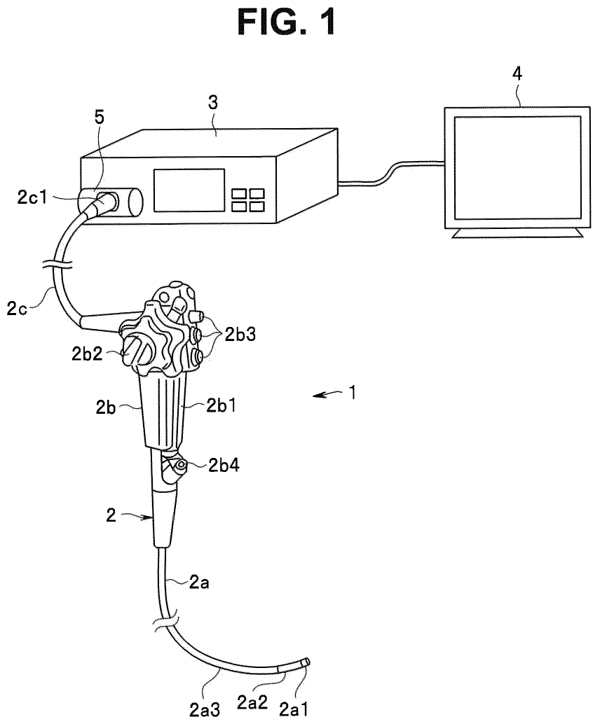

FIG. 1 to FIG. 7 show a first embodiment of the present disclosure. FIG. 1 is a view showing a configuration of an endoscope system 1 in the first embodiment.

The endoscope system 1 includes an endoscope 2, an endoscope processor 3, a monitor 4, and a relay adapter 5.

Note that in the present embodiment, the endoscope system 1 as a medical device system, the endoscope 2 as a medical device, and the endoscope processor 3 as a power supply device that supplies power to the medical device are respectively used as examples. However, the medical device system is not limited to the endoscope system 1. For example, the medical device may be a treatment instrument such as a catheter. Therefore, the power supply device is not limited to the endoscope processor 3.

The endoscope 2 is a single-use endoscope that is disposed of after single use, for example. However, the endoscope 2 is not limited to the single-use endoscope.

Meanwhile, the endoscope processor 3 is a reusable endoscope processor used for a reusable endoscope, for example.

The single-use endoscope may have a different connector standard (a hardware standard such as a connector shape, and a software standard such as a signal standard) from that of the reusable endoscope. Therefore, the relay adapter 5 is a configuration that enables the single-use endoscope, whose connector standard differs from that of the reusable endoscope, to be connected to the endoscope processor 3.

Note that the endoscope connected to the endoscope processor 3 using the relay adapter 5 is not limited to a single-use endoscope, and may be another reusable endoscope whose connector standard differs from that of the reusable endoscope that can be connected to the endoscope processor 3.

The relay adapter 5 emulates the reusable endoscope that can be connected to the endoscope processor 3. The endoscope processor 3 thus recognizes the single-use endoscope 2 connected through the relay adapter 5 as an endoscope having a specification capable of being connected to the endoscope processor 3, and performs image processing or other processing. The relay adapter 5 may be single use, but is preferably assumed to be reusable.

The endoscope 2 includes an insertion portion 2a, an operation portion 2b, and a universal cable 2c. The endoscope 2 is configured as an electronic endoscope, for example.

The insertion portion 2a is a part to be inserted into a subject. The subject is assumed to be a living object such as a human or an animal, for example. However, the subject may also be a non-living object such as a machine or a building.

The insertion portion 2a includes a distal end portion 2a1, a bending portion 2a2, and a flexible tube portion 2a3 in this order from a distal end side to a proximal end side.

In the distal end portion 2a1, an image pickup unit, a distal end portion of a light guide, and a distal end side opening of a treatment instrument channel, and the like are arranged. The image pickup unit includes an image pickup optical system and an image sensor. The image pickup optical system includes a plurality of lenses and an optical aperture, and the like, and forms an optical image of the subject on the image sensor. The image sensor photoelectrically converts (picks up) the optical image of the subject to generate an image pickup signal.

Examples of the image sensor are a CCD (charge coupled device) image sensor, a CMOS (complementary metal-oxide semiconductor) image sensor, or the like, but are not limited to those.

The bending portion 2a2 is a part than can be bent in two directions (up and down) or four directions (up, down, left, and right), for example.

The flexible tube portion 2a3 is a tube portion having a flexibility. Note that the following is an example where the endoscope 2 is a flexible endoscope with the flexible tube portion 2a3. However, the endoscope 2 may be a rigid endoscope in which a portion corresponding to the flexible tube portion 2a3 is a rigid form.

The operation portion 2b is a part for operating the endoscope 2 by a user. The operation portion 2b is disposed on a proximal end side of the insertion portion 2a. The operation portion 2b includes a grasping portion 2b1, a bending operation knob 2b2, a plurality of operation buttons 2b3, and a treatment instrument insertion port 2b4.

The grasping portion 2b1 is a part where the user grasps the endoscope 2 with a palm.

The bending operation knob 2b2 is an operation device for operating bending of the bending portion 2b2. The bending operation knob 2b2 is operated by using a thumb of a hand grasping the grasping portion 2b1, for example. When the bending operation knob 2b2 is operated, a bending wire is pulled and the bending portion 2a2 is bent.

When the bending portion 2a2 is bent, a direction of the distal end portion 2a1 changes. Then an image pickup direction by the image pickup unit and an irradiation direction of an illumination light by the light guide change. Furthermore, the bending portion 2a2 is bent for improving insertability of the insertion portion 2a in the subject.

The plurality of operation buttons 2b3 includes gas/liquid feeding buttons, a suction button, and other buttons, for example. The gas/liquid feeding buttons are buttons for operating gas/liquid feeding to an observation window provided in a distal end surface of the image pickup unit at the distal end portion 2a1. The observation window is cleaned by the liquid feeding and the liquid after cleaning is blown by the gas feeding. The gas/liquid feeding is performed through a not-shown gas/liquid feeding channels.

The suction button is a button for operation to suction liquids, mucus membrane, or the like in the subject from the distal end portion 2a1. For example, the liquids, the mucus membrane, or the like are suctioned through a suction channel provided in the endoscope 2.

Other buttons are a freeze button to pause a monitor screen, a release button to pick up a still image, a switch button to a special light, and the like, for example.

The treatment instrument insertion port 2b4 is an opening on a proximal end side of the treatment instrument channel. A treatment instrument such as forceps is inserted in the treatment instrument channel from the treatment instrument insertion port 2b4. A distal end portion of the treatment instrument protrudes from a distal end side opening of the treatment instrument channel. Various treatments are performed to the subject by the protruded distal end portion of the treatment instrument.

The universal cable 2c is extended from the operation portion 2b. An extended end of the universal cable 2c is provided with a first connector 2c1.

As described above, the first connector 2c1 of the single-use endoscope 2 is different in standard from the connector of the reusable endoscope.

The single-use endoscope 2 is connected to the endoscope processor 3 through the relay adapter 5.

The endoscope processor 3 supplies power to the endoscope 2 through the relay adapter 5, and transmits a control signal to the endoscope 2. Furthermore, the endoscope processor 3 receives an image pickup signal from the endoscope 2 through the relay adapter 5.

The endoscope processor 3 performs image processing on the image pickup signal received from the endoscope 2 to generate a video signal. The generated video signal is outputted to the monitor 4 to cause an endoscope image to be displayed on the monitor 4.

Note that the following description will be made as the endoscope processor 3 of the present embodiment supplies an illumination light to the endoscope 2 through the relay adapter 5. However, a light source apparatus may be provided separately from the endoscope processor 3, or a configuration in which a light emitting device such as an LED (light-emitting diode) may be provided in the distal end portion of the endoscope 2 to emit an illumination light may be adopted.

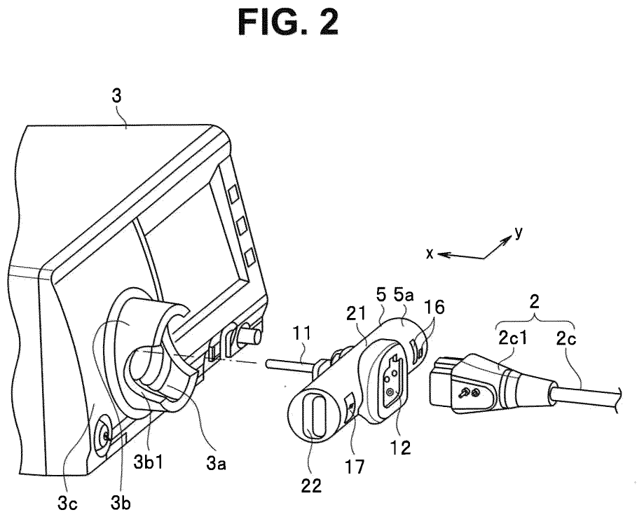

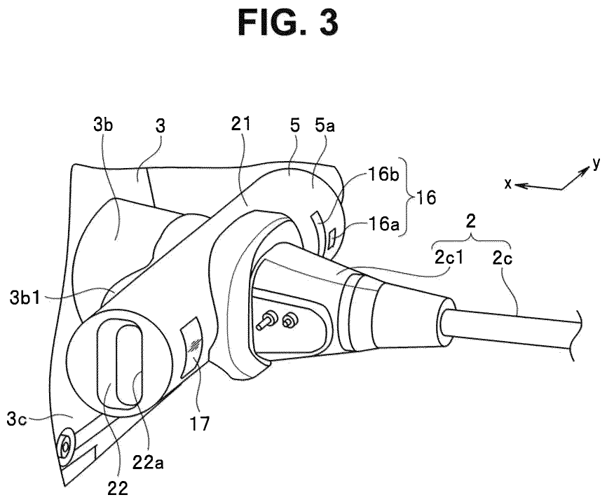

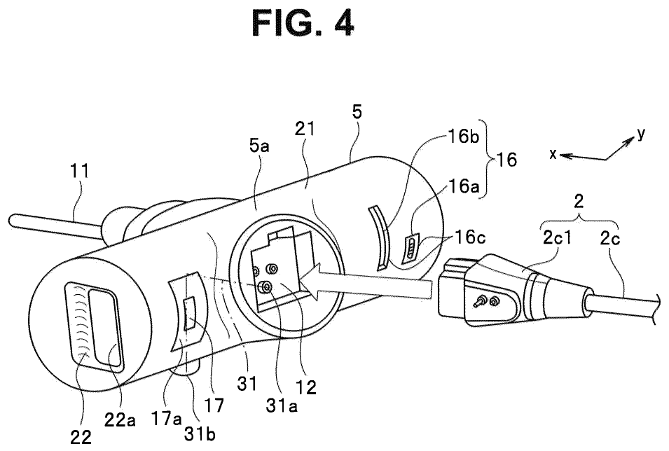

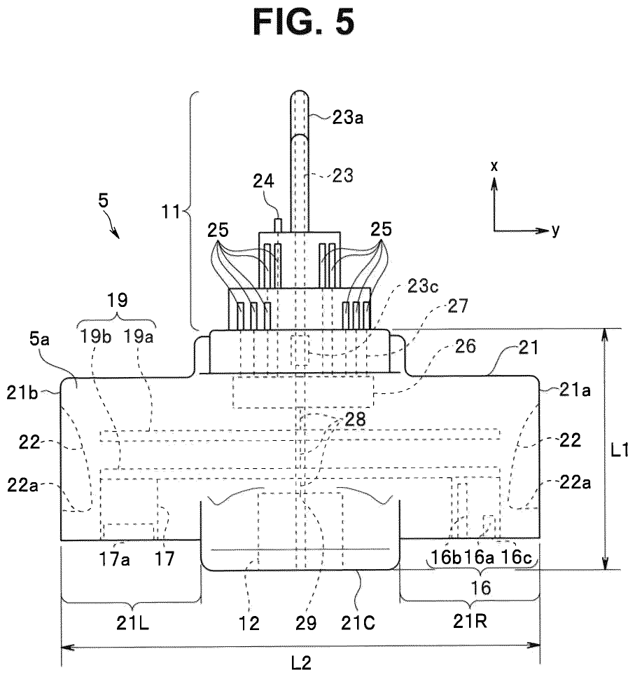

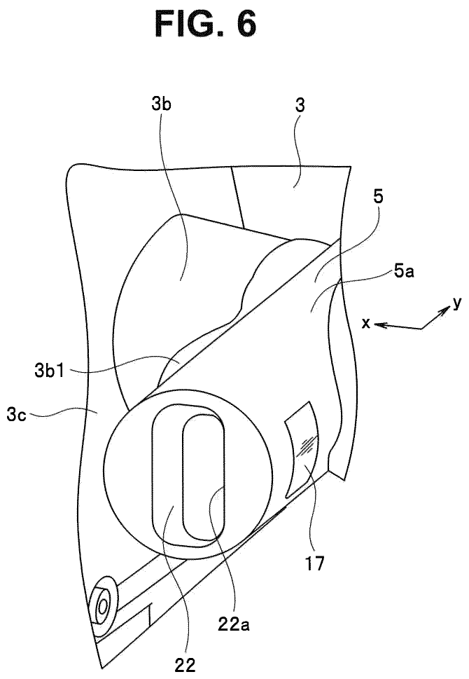

FIG. 2 is a partial exploded perspective view showing a configuration in which the endoscope 2 and the endoscope processor 3 are connected using the relay adapter 5 in the first embodiment. FIG. 3 is a partial perspective view showing a state in which the endoscope 2 and the endoscope processor 3 are connected using the relay adapter 5 in the first embodiment. FIG. 4 is a perspective view showing the relay adapter 5 and the first connector 2c1 of the endoscope 2 in the first embodiment. FIG. 5 is a plan view showing a configuration of the relay adapter 5 in the first embodiment. FIG. 6 is a partial enlarged perspective view showing a configuration when the relay adapter 5 of the first embodiment is connected to a first receptacle 3a of the endoscope processor 3.

As shown in FIG. 2 to FIG. 6, a direction from the relay adapter 5 toward the endoscope processor 3 (a direction in which a second connector 11 of the relay adapter 5 is inserted into the first receptacle 3a of the endoscope processor 3) is an x direction (first direction), and a direction intersecting the first direction is a second direction. In the present embodiment in particular, a direction orthogonal to the x direction is a y direction. The y direction is a direction to the right when viewing at the front of the endoscope processor 3 when the endoscope processor 3 is placed, for example.

The endoscope processor 3 includes the first receptacle 3a. The first receptacle 3a is connected to a connector of the reusable endoscope. The first receptacle 3a is a connector receiver with a standard corresponding to a standard of the connector of the reusable endoscope.

The endoscope processor 3 includes a wall-shaped part 3b (protrusion) that protrudes in a wall-shape from a surface (exterior surface) of an exterior member 3c of the endoscope processor 3 around the first receptacle 3a. The wall-shaped part 3b includes cutout parts 3b1 cut out such that a protruded amount from the exterior surface of the endoscope processor 3 becomes smaller. The cutout parts 3b1 are provided in +y direction and −y direction of the wall-shaped part 3b, respectively.

The wall-shaped part 3b surrounds at least part of a side surface of the relay adapter 5 connected to the first receptacle 3a (see FIG. 3, FIG. 6, and the like).

The relay adapter 5 includes a main body 5a, and a second connector 11 (first connector) extending from the main body 5a in the x direction (first direction). The second connector 11 is detachably connected to the first receptacle 3a of the endoscope processor 3.

The second connector 11 is a connector with a standard corresponding to the standard of the connector of the reusable endoscope to which the endoscope processor 3 can connect.

The main body 5a includes a second receptacle 12. The first connector 2c1 (second connector) has a shape that cannot be connected to the first receptacle 3a and can be connected to the second receptacle 12. The first connector 2c1 of the endoscope 2 is detachably connected to the second receptacle 12.

The second receptacle 12 is a connector receiver with a standard corresponding to a standard of the first connector 2c1 of the single-use endoscope 2.

The main body 5a includes an exterior member 21 (lateral body). The exterior member 21 extends in the second direction. Specifically, as shown in FIG. 5, the exterior member 21 is provided with the second receptacle 12 at a center part 21C, and the second connector 11 is extended from the center part 21C in the +x direction. In the exterior member 21, a right extension part 21R (first lateral portion) is extended in the the +y direction and a left extension part 21L (second lateral portion) is extended in the −y direction, from the center part 21C. In other words, the right extension part 21R is at a first side of the exterior member 21 and the left extension part 21L is at a second side of the exterior member 21. The first side and the second side are on opposite sides of an intersection portion that the axis extending in the second direction intersecting an axis of the second connector 11.

The second direction in which the exterior member 21 extends is a direction not in contact with the wall-shaped part 3b, that is, a direction of the cutout part 3b1. In this state, the relay adapter 5 can be stably held to the wall-shaped part 3b by configuring an outer shape of the exterior member 21 to fit the cutout part 3b1.

That is, in the relay adapter 5, the second connector 11 extends from the middle of the main body 5a in an axial direction (y direction), and the entire the main body 5a and the second connector 11 form a T-shape.

As shown in FIG. 5, for example, an entire length L2 of the exterior member 21 in the y direction is longer than an entire length L1 of the exterior member 21 in the x direction. When the relay adapter 5 is connected to the endoscope processor 3, the second connector 11 of the relay adapter 5 is roughly housed in the first receptacle 3a. Therefore, when the relay adapter 5 is connected to the endoscope processor 3, a length that the relay adapter 5 protrudes from a surface of the exterior member 3c in the −x direction is roughly the entire length L1 of the exterior member 21 in the x direction.

By making the entire length L2 of the exterior member 21 in the y direction longer than the entire length L1 in the x direction, a protrusion length from an exterior surface can be shortened compared to a conventional relay adapter while securing a sufficient space in the exterior member 21.

The main body 5a includes a grip 22 provided at least one of in an end surface of the exterior member 21 in the second direction (axial direction) or between the second receptacle 12 and the end surface. The grip 22 is a recessed-shape portion, for example, and is grasped when the second connector 11 is removed from the first receptacle 3a. Note that an example is shown here where the grip 22 is the recessed-shape portion, but the grip 22 may be a convex shape portion, a ring, or a hook.

The grip 22 includes a plurality of grips 22. The plurality of grips 22 are respectively provided in an end surface 21a (first end surface) in the +y direction and an end surface 21b (second end surface) in the −y direction of the exterior member 21 (both end surfaces 21a, 21b of the exterior member 21 protruding from the wall-shaped part 3b) when the relay adapter 5 is connected to the endoscope processor 3. The grip 22 includes a finger hook surface 22a used when the second connector 11 is removed from the first receptacle 3a.

Since the relay adapter 5 of the present embodiment is provided with the grips 22, a user can easily remove the second connector 11 from the first receptacle 3a even if the protrusion length from the exterior surface is shorter compared to the conventional relay adapter.

As shown in FIG. 5, the main body 5a includes an electrical circuit substrate 19 disposed inside the exterior member 21. As described above, in the exterior member 21, the entire length L2 in the y direction is longer than the entire length L1 in the x direction. The electrical circuit substrate 19 is arranged in the y direction in the exterior member 21, extending across the left extension part 21L, the center part 21C, and the right extension part 21R. This allows for a larger area of the electrical circuit substrate 19, and more circuits can be configured on the electrical circuit substrate 19.

In the example of FIG. 5, the electrical circuit substrate 19 includes a first substrate 19a and a second substrate 19b. However, the number of substrates is not limited to two, but may be one, or may be three or more.

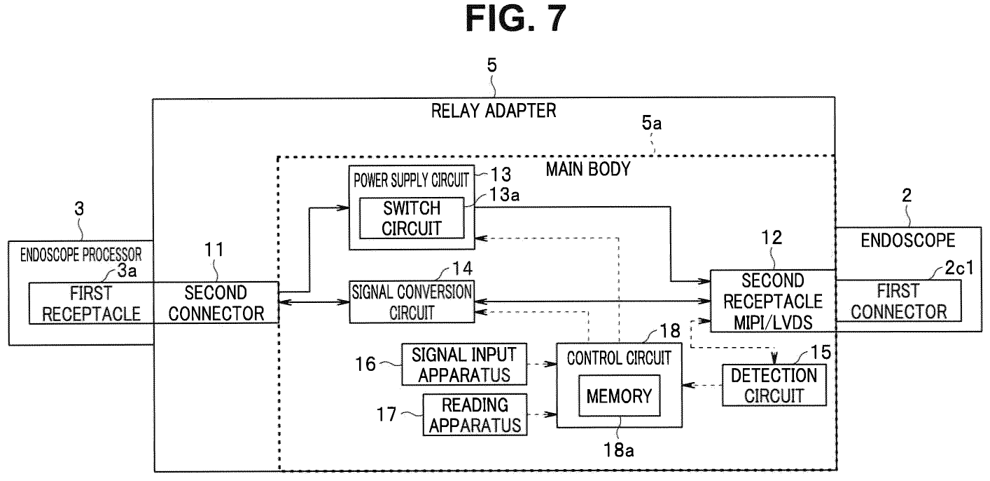

FIG. 7 is a block diagram showing an electrical configuration of the relay adapter 5 in the first embodiment.

The main body 5a of the relay adapter 5 includes a power supply circuit 13, a signal conversion circuit 14, a detection circuit 15, a signal input apparatus 16, a reading apparatus 17, and a control circuit 18, in addition to the second receptacle 12.

Each circuit of the relay adapter 5 can operate when the second connector 11 is connected to the first receptacle 3a.

In the electrical circuit substrate 19 shown in FIG. 5, the power supply circuit 13, the signal conversion circuit 14, the detection circuit 15, and the control circuit 18 are configured, for example.

The control circuit 18 includes a memory 18a. The memory 18a includes a rewritable non-volatile memory, for example. Note that in the example of FIG. 7, the memory 18a is provided in the control circuit 18, but the memory 18a may be provided separately from the control circuit 18.

The control circuit 18 is configured to perform each function by a processor such as an ASIC (application specific integrated circuit), an FPGA (field programmable gate array), or the like, which includes a CPU (central processing unit) or the like, reading and executing a processing program stored in a storage unit (or recording medium) such as the memory 18a, for example. However, the each function of the control circuit 18 may be configured to be performed by a dedicated electronic circuit.

The memory 18a stores information for emulating the single-use endoscope 2 connected to the relay adapter 5 to the reusable endoscope that can be connected to the endoscope processor 3.

Here, there may be a plurality of models of the single-use endoscope 2, and there may be a plurality of models of reusable endoscopes that can be connected to the endoscope processor 3. Therefore, the memory 18a stores information for emulating one or more models of the single-use endoscopes 2 to one or more models of reusable endoscopes.

Specifically, the memory 18a stores ID information (model number indicating the model, serial number for distinguish an individual, or the like) and information on the standard or the like of one or more models of the reusable endoscopes that can be connected to the endoscope processor 3. In addition, the memory 18a stores the ID information, the information on the standard or the like of one or more models of the single-use endoscopes.

Furthermore, the memory 18a stores setting information, as a table for example, according to a combination of the model (ID information) of the single-use endoscope and the model (ID information) of the reusable endoscope. Note that in a case of a configuration in which the relay adapter 5 emulates only one model of the reusable endoscope, the memory 18a may store a table according to the model of the single-use endoscope.

The setting information stored in the table in the memory 18a includes information for setting the power supply circuit 13 and information for setting the signal conversion circuit 14.

The control circuit 18 also serves as a communication unit that communicates with the endoscope 2 and the endoscope processor 3. The control circuit 18 also serving as the communication unit absorbs a difference in communication with the endoscope 2 and the endoscope processor 3 to allow for making communication in common.

The single-use endoscope 2 generally includes a memory to store the ID information (further setting value, information on the image sensor, or the like, as needed), and a circuit to transmit the ID information or the like. The single-use endoscope 2 should be disposed of after a single use and should not be used multiple times as described above. Therefore, the memory of the endoscope 2 may store information such as an energization time, date of start of use, or the like. Based on these information, the endoscope system 1 may prohibit reuse of the endoscope 2.

The control circuit 18 functions as the communication unit and acquires the ID information or the like from the single-use endoscope 2.

In a case where the relay adapter 5 can emulate a plurality of models of the reusable endoscopes, the control circuit 18 refers to the table in the memory 18a according to the ID information of the endoscope 2 and determines the one model of the reusable endoscope most suitable for emulation as a target of the emulation.

The control circuit 18 transmits the ID information or the like on the reusable endoscope that is determined to be emulated to the endoscope processor 3. The endoscope processor 3 thus judges that the reusable endoscope to be emulated is connected when the endoscope 2 is connected through the relay adapter 5. The endoscope processor 3 then supplies power and a control signal that are compatible with the reusable endoscope to be emulated to the relay adapter 5.

Furthermore, the control circuit 18 acquires setting information for emulating the endoscope 2 to the determined one model of the reusable endoscope from the table in the memory 18a.

The control circuit 18 functions as a setting unit, and sets the power supply circuit 13 and the signal conversion circuit 14 based on the acquired setting information.

Here, it is assumed that voltages of power lines compatible with the reusable endoscope to be emulated are, for example, one of 1.2 V and two of 1.8 V. On the other hand, it is assumed that voltages of power lines compatible with the single-use endoscope 2 are, for example, two of 1.0 V and one of 1.2 V.

The control circuit 18 then sets the power supply circuit 13 to convert voltages of input lines of one of 1.2 V and two of 1.8 V into voltages of output lines of two of 1.0 V and one of 1.2 V.

The power supply circuit 13 supplies the power transmitted from the endoscope processor 3 to the endoscope 2.

Specifically, the endoscope processor 3 supplies power compatible with the reusable endoscope to be emulated. The power supply circuit 13 converts the power (voltages, currents, the number, or the like) transmitted from the endoscope processor 3 to power compatible with the single-use endoscope 2 according to the setting. The converted power is transmitted to the first connector 2c1 of the endoscope 2 through the second receptacle 12.

The control circuit 18 further sets the signal conversion circuit 14 based on the acquired setting information. The signal conversion circuit 14 then converts the signal transmitted from the endoscope processor 3 into a signal compatible with the endoscope 2 and transmits the converted signal to the endoscope 2. Furthermore, the signal conversion circuit 14 converts a signal transmitted from the endoscope 2 into a signal compatible with the endoscope processor 3 and transmits the converted signal to the endoscope processor 3.

Specifically, the signal conversion circuit 14 converts control signals (control signals such as a clock signal, a vertical synchronization signal, and a horizontal synchronization signal that are transmitted toward the reusable endoscope) transmitted from the endoscope processor 3 into control signals compatible with the endoscope 2 and transmits the converted control signals to the endoscope 2. The image sensor equipped with the endoscope 2 then generates an image pickup signal and transmits the image pickup signal to the signal conversion circuit 14.

Furthermore, the control circuit 18 communicates with the endoscope processor 3 and acquires information on white balance setting from the endoscope processor 3. The control circuit 18 controls the signal conversion circuit 14 based on the information on the white balance setting to perform white balance adjustment. Information required to perform the white balance adjustment by the signal conversion circuit 14 is stored in the memory 18a. This eliminates the need for a circuit to perform the white balance adjustment, thus reducing a price of the endoscope 2.

Furthermore, by separating information to be stored in the memory of the endoscope 2 and information to be stored in the memory 18a of the relay adapter 5, a storage capacity of the memory of the endoscope 2 can be reduced. This further reduces the price of the endoscope 2.

The signal conversion circuit 14 converts signals such that a format (the number of pixels, frame rate, or the like) of the image pickup signals generated by the image sensor equipped with the single-use endoscope 2 becomes a format of image pickup signals generated by the image sensor equipped with the reusable endoscope of the target of the emulation. The signal conversion circuit 14 transmits the converted image pickup signals to the endoscope processor 3.

The detection circuit 15 operates when the relay adapter 5 is connected to the endoscope processor 3, and detects whether the second receptacle 12 is connected to the first connector 2c1 (that is, whether the endoscope 2 is connected to the relay adapter 5).

The detection circuit 15 detects a presence or absence of a connection according to whether a contact for detection of the second receptacle 12 and a contact for detection of the first connector 2c1 are energized, for example. However, the detection circuit 15 may also detect the presence or absence of the connection using other known technologies as appropriate. The detection circuit 15 transmits a detection result to the control circuit 18.

The power supply circuit 13 includes a switch circuit 13a that is controlled by the control circuit 18.

The control circuit 18 causes the switch circuit 13a to stop supplying power from the power supply circuit 13 to the endoscope 2 when the detection circuit 15 detects that the second receptacle 12 is not connected to the first connector 2c1. This can prevent current from flowing through an electrical contact of the second receptacle 12.

Furthermore, the control circuit 18 allows the switch circuit 13a to supply power from the power supply circuit 13 to the endoscope 2 when the detection circuit 15 detects that the second receptacle 12 is connected to the first connector 2c1.

Thus, by providing the switch circuit 13a, a safety of the relay adapter 5 is enhanced and malfunctions can be prevented.

Note that an example where the switch circuit 13a is provided in the power supply circuit 13 is shown in FIG. 7, the switch circuit 13a may be provided outside the power supply circuit 13, as long as it is on a supply path of power in the relay adapter 5.

For example, the image sensor mounted in the single-use endoscope 2 may be updated to manufacture and sale a new model. The relay adapter 5 may be configured to supply various −ypes of power to an extent that it is expected for a new model of the single-use endoscope 2.

Meanwhile, when developing a new model of the single-use endoscope 2, an image sensor to an extent that the relay adapter 5 can supply power may be adopted for the endoscope 2.

The table stored in the rewritable memory 18a then is updated to a table corresponding to the ID information of the new model of the endoscope 2.

This allows the relay adapter 5 to supply power to the new model of the endoscope 2 based on the ID information simply by upgrading a version of software without changing hardware. Therefore, it is no longer necessary to provide the relay adapter 5 for each model of the endoscope 2, reducing an introduction cost for a user.

When the price of the endoscope 2 is lowered, a general-purpose connector and a general-purpose signal standard may be used for the first connector 2c1, for example. Specifically, the first connector 2c1 is configured as a USB (universal serial bus) connector, for example, and a transmission standard for camera (MIPI: mobile industry processor interface) is used as a signal standard, for example. By using the MIPI, an update of the image sensor can be easily handled, for example.

The second receptacle 12 is configured as a USB receptacle corresponding to the first connector 2c1, for example, and the MIPI is used as a signal standard.

Note that the signal standard for the first connector 2c1 and the second receptacle 12 is not limited to the MIPI, LVDS (low voltage differential signaling) or the like may be used.

The general-purpose connector and the general-purpose signal standard may continue to be used for a new model with an updated image sensor of the endoscope 2.

With this, when a different model of the single-use endoscope 2 is connected to the endoscope processor 3 after a certain model of the single-use endoscope 2 is connected to the endoscope processor 3, the relay adapter 5 no longer needs to be replaced, eliminating user's time and effort.

The relay adapter 5 includes a light guide body 23 to transmit an illumination light. A part of the light guide body 23 on a +x direction side protrudes as a light guide portion 23a in the second connector 11. The light guide body 23 transmits to the second receptacle 12 an illumination light supplied from the endoscope processor 3 to the light guide portion 23a of the second connector 11.

The relay adapter 5 includes an optical shutter 23c on an optical path of the illumination light transmitted by the light guide body 23.

The optical shutter 23c blocks the illumination light transmitted from the second connector 11 toward the second receptacle 12 based on control by the control circuit 18 when the detection circuit 15 detects that the second receptacle 12 is not connected to the first connector 2c1.

Furthermore, the optical shutter 23c allows the illumination light transmitted from the second connector 11 toward the second receptacle 12 to path through based on the control by the control circuit 18 when the detection circuit 15 detects that the second receptacle 12 is connected to the first connector 2c1.

In the electrical circuit substrate 19, the reading apparatus 17 to read external code information (two-dimensional code information, for example) is mounted. The reading apparatus 17 is configured as a barcode reader, for example. The reading apparatus 17 reads the external code information via a sensor (from a window 17a) provided in the exterior member 21.

Note that the barcode reader is used here as an example of the reading apparatus 17, but the known technologies such as an RFID (radio frequency identification) may be used as appropriate.

In the electrical circuit substrate 19, the signal input apparatus 16 into which an external signal is inputted is mounted. The signal input apparatus 16 receives the external signal from a window 16c provided in the exterior member 21. In the examples shown in FIG. 3 to FIG. 5, the signal input apparatus 16 includes a first signal input apparatus 16a and a second signal input apparatus 16b.

The first signal input apparatus 16a is configured as an electronic device connector receiver, for example. Specifically, the first signal input apparatus 16a may be configured as a USB receptacle to receive a signal through a USB cable.

The second signal input apparatus 16b is configured as an SD (secure digital) card reader, for example.

The signal input apparatus 16 is not limited to these examples, but other known technologies may be used as appropriate.

The signal input apparatus 16 and the reading apparatus 17 are used for upgrading a version of software (including the above described table and the like) stored in the memory 18a, for example. By upgrading the version of the software, the relay adapter 5 can handle a new model of the single-use endoscope 2.

For example, in a state where the relay adapter 5 is connected to a network by the first signal input apparatus 16a or a not-shown wireless communication apparatus, two-dimensional code information or the like for upgrading the version is read by the reading apparatus 17. Then the relay adapter 5 may be automatically connected to a network site for upgrading the version so that upgrading the version of the software is automatically performed.

Furthermore, the endoscope 2 may be provided with two-dimensional code information or the like for recording image sensor information. In this case, the reading apparatus 17 reads the two-dimensional code information or the like of the endoscope 2 to acquire the image sensor information or the like.

As described above, the electrical circuit substrate 19 is arranged to extend in the y direction across the left extension part 21L, the center part 21C, and the right extension part 21R. For this, in the present embodiment, the electrical circuit substrate 19 allows more room for circuit arrangement, and can mount the reading apparatus 17, the first signal input apparatus 16a, and the second signal input apparatus 16b.

However, the electrical circuit substrate 19 may only include any of one or more of the reading apparatus 17, the first signal input apparatus 16a, and the second signal input apparatus 16b. Furthermore, if there is not enough space in the exterior member 21, the signal input apparatus 16 and the reading apparatus 17 may not be provided.

The second connector 11 includes an electrical contact 24 and a plurality of electrical contacts 25. The main body 5a includes an internal electrical connector 26 on the +x direction side of the electrical circuit substrate 19. The electrical contact 24 and the plurality of electrical contacts 25 are connected to the internal electrical connector 26 through signal lines 27, respectively. The internal electrical connector 26 is connected to the electrical circuit substrate 19 through a signal line 28.

Accordingly, the internal electrical connector 26 collectively connects the plurality of signal lines 27 extending from the electrical contact 24 and the plurality of electrical contacts 25 to the electrical circuit substrate 19.

The second receptacle 12 includes an electrical contact 29. The signal line 28 further connects the electrical circuit substrate 19 and the electrical contact 29. The electrical contact 29 is connected to an electrical contact of the first connector 2c1.

The relay adapter 5 includes a fluid conduit 31 as shown in FIG. 4. The fluid conduit 31 includes a first fluid connector 31a on one end side and a second fluid connector 31b on another end side. The first fluid connector 31a is arranged in the second receptacle 12, for example. The second fluid connector 31b is provided to protrude from the exterior member 21, for example. The second fluid connector 31b is provided at a position above the exterior member 21 outside of the wall-shaped part 3b when the second connector 11 and the first receptacle 3a are connected.

The first fluid connector 31a is connected to a fluid conduit of the endoscope 2. The second fluid connector 31b is connected to a fluid conduit of a not-shown fluid apparatus (or a fluid conduit of the endoscope processor 3). The fluid conduit 31 distributes fluid between the endoscope processor 3 and the fluid conduit of the endoscope 2.

Note that not limited to the configuration shown in FIG. 4, the second fluid connector 31b may be provided in the second connector 11 to be connected to the fluid conduit of the endoscope processor 3.

Furthermore, in the endoscope system 1, a plurality −ypes of fluid such as fluid for gas/liquid feeding, and fluid of suction may be handled. Therefore, the relay adapter 5 may include both of the second fluid connector 31b protruding from the exterior member 21 and the second fluid connector 31b arranged in the second connector 11.

According to the first embodiment, the exterior member 21 of the relay adapter 5 extends in the second direction (y direction) intersecting the first direction (x direction) so that the entire the main body 5a and the second connector 11 form the T-shape. Therefore, the protrusion of the relay adapter 5 from the endoscope processor 3 can be suppressed compared to the conventional relay adapter.

Thus in a case where the endoscope chamber is narrow, or the like, the relay adapter 5, which remains inserted in the first receptacle 3a of the endoscope processor 3, can be prevented from restricting the operative space.

Furthermore, by remaining the relay adapter 5 inserted in the first receptacle 3a of the endoscope processor 3, even when the endoscope 2 is used for many cases, the number of times the relay adapter 5 is attached to and detached from the first receptacle 3a can be reduces. Therefore, deterioration of the first receptacle 3a can be suppressed.

Since the second direction (y direction) in which the exterior member 21 extends is a direction in which the cutout part 3b1 of the wall-shaped part 3b is provided, when the relay adapter 5 is attached to or detached from the first receptacle 3a, the wall-shaped part 3b avoids becoming an obstacle to attaching and detaching.

The grips 22 are provided in at least one end surface (both end surfaces 21a and 21b in the −y direction and the-y direction in the present embodiment) of the exterior member 21 in the y direction. Therefore, even if the protrusion of the relay adapter 5 from the endoscope processor 3 in the −x direction is small, the user can easily attach and detach (including removal) the relay adapter 5 to and from the endoscope processor 3.

At this time, since the finger hook surface 22a is provided in each of the grips 22, the removal of the relay adapter 5 becomes easier by hooking the finger of the user on the surface 22a.

By providing the exterior member 21 that extends in the y direction, there is more space in the exterior member 21, and the electrical circuit substrate 19 with a larger area than in conventional relay adapters can be arranged. This allows each circuit, each apparatus, or the like shown in FIG. 7 to be housed in the exterior member 21, making it possible to configure the highly functional relay adapter 5.

For example, by providing the signal input apparatus 16, the reading apparatus 17, and the like, the version of the software can be upgraded, making the relay adapter 5 more scalable.

Since the control circuit 18 functioning as the setting unit sets the power supply circuit 13 and the signal conversion circuit 14 according to the model or the like of the endoscope 2, compatibility of the endoscope 2 with the endoscope processor 3 can be ensured.

Since the relay adapter 5 includes the power supply circuit 13, it is no longer necessary for the single-use endoscope 2 to include a circuit that converts power supplied from the endoscope processor 3. This simplifies the configuration of the endoscope 2 and further lowers the price of the endoscope 2.

Thus, the relay adapter 5 of the present embodiment can lower the barrier to the developing the new model of the single-use endoscopes 2 and facilitate the introduction of the single-use endoscopes 2 to the market.

Furthermore, in a facility that is already equipped with the endoscope processor 3 for the reusable endoscope and wishes to newly use the single-use endoscope 2, it is no longer necessary to purchase an endoscope processor dedicated to the single-use endoscope 2, reducing an initial investment of a user.

By using a general-purpose connector and a general-purpose signal standard for the first connector 2c1 of a plurality models of the endoscopes 2 to configure the second receptacle 12 to be the standard corresponding to the first connector 2c1, a relay adapter for each model becomes unnecessary. This makes it possible to use a common relay adapter 5 for a plurality of models of the endoscopes 2, and thus realize the simpler endoscope system 1.

Note that the present disclosure is not limited to the embodiment described above as it is. The present disclosure can be embodied by modifying the constituent elements at the implementation stage to the extent that does not depart from the gist of the disclosure. In addition, a plurality of constituent elements disclosed in the above embodiment can be combined as appropriate to form various aspects of the disclosure. For example, some constituent elements may be deleted from the all constituent elements disclosed in the embodiment. Furthermore, the constituent elements of different embodiments may be combined as appropriate. Thus, it is obviously to make various variations and applications within the scope that does not depart from the gist of the disclosure.

What is claimed is:

1. A relay adapter, comprising:
a first connector extending in a first direction, the first connector configured to be connected to a first receptacle of a power supply device; and
a main body including:
a lateral body extending in a second direction intersecting the first direction,
a second receptacle configured to be connected to a second connector of a medical device, and
a grip provided at least one of (i) in an end surface of the lateral body in the second direction or (ii) between the second receptacle and the end surface.

2. The relay adapter according to claim 1, wherein an entire length of the lateral body in the second direction is longer than an entire length of the lateral body in the first direction.

3. The relay adapter according to claim 2, wherein:
the main body further includes an electrical circuit substrate disposed inside the lateral body,
the electrical circuit substrate includes a power supply circuit and a signal conversion circuit,
the power supply circuit is configured to supply power transmitted from the power supply device to the medical device, and
the signal conversion circuit is configured to:
convert a first signal transmitted from the power supply device into a first converted signal compatible with the medical device and transmit the first converted signal to the medical device, and
convert a second signal transmitted from the medical device into a second converted signal compatible with the power supply device and transmit the second converted signal to the power supply device.

4. The relay adapter according to claim 3, wherein the lateral body includes a sensor, and
wherein the electrical circuit substrate is mounted with a reading apparatus being configured to read external code information via the sensor.

5. The relay adapter according to claim 3, wherein the lateral body includes a sensor, and
wherein the electrical circuit substrate is mounted with a signal input apparatus being configured to receive an external signal via the sensor.

6. The relay adapter according to claim 3, wherein the electrical circuit substrate further includes:
a detection circuit configured to detect whether the second connector is connected to the second receptacle, and
a switch circuit configured to stop supplying power from the power supply circuit to the medical device when the detection circuit detects that the second connector is not connected to the second receptacle.

7. The relay adapter according to claim 6, further comprising a light guide body configured to receive an illumination light supplied from the power supply device via the first connector and to transmit the illumination light from the first connector to the second receptacle.

8. The relay adapter according to claim 7, further comprising an optical shutter configured to block the illumination light transmitted from the first connector to the second receptacle when the detection circuit detects that the second connector is not connected to the second receptacle.

9. The relay adapter according to claim 1, wherein:
the lateral body includes:
a first lateral portion extending in the second direction, the first lateral portion being at a first side of the lateral body, and
a second lateral portion extending opposite to the first lateral portion in the second direction with respect to the second receptacle, the second lateral portion being at a second side of the lateral body,
the second direction intersects an axis of the first connector at an intersection portion, and
the first side and the second side are on opposite sides of the intersection portion.

10. The relay adapter according to claim 9, wherein the grip includes a plurality of grips, the plurality of grips includes a first grip in a first end surface of the first lateral portion and a second grip in a second end surface of the second lateral portion.

11. The relay adapter according to claim 1, wherein the grip is recessed-shape portion.

12. The relay adapter according to claim 10, wherein the first grip and the second grip each include a finger hook surface.

13. The relay adapter according to claim 1, wherein the medical device is a single-use endoscope.

14. A medical device system, comprising:
a medical device including a connector;
a power supply device including a first receptacle, the power supply device configured to transmit power to be supplied to the medical device; and
a relay adapter including:
a first connector extending in a first direction, the first connector configured to be connected to the first receptacle, and
a main body including:
a lateral body extending in a second direction intersecting the first direction,
a second receptacle configured to be connected to the connector of the medical device, and
a grip provided at least one of (i) in an end surface of the lateral body in the second direction or (ii) between the second receptacle and the end surface.

15. The medical device system according to claim 14, wherein an entire length of the lateral body in the second direction is longer than an entire length of the lateral body in the first direction.

16. The medical device system according to claim 15, wherein:
the power supply device includes a protrusion located around at least a portion of a periphery of the first receptacle, the protrusion extending outward from an exterior surface of the power supply device,
the protrusion includes a cutout, a protruding length of the cutout from the exterior surface being a first length, a protruding length of the protrusion in a region other than the cutout from the exterior surface being a second length, the first length being smaller than the second length, a direction of the protruding length of the cutout being the second direction, and
the protrusion surrounds at least a portion of a side surface of the relay adapter when the first connector is connected to the first receptacle.

17. The medical device system according to claim 15, wherein:
the main body further includes an electrical circuit substrate disposed inside the lateral body,
the electrical circuit substrate includes a power supply circuit and a signal conversion circuit,
the power supply circuit is configured to supply power transmitted from the power supply device to the medical device, and the signal conversion circuit is configured to:
convert a first signal transmitted from the power supply device into a first converted signal compatible with the medical device and transmit the first converted signal to the medical device, and
convert a second signal transmitted from the medical device into a second converted signal compatible with the power supply device and transmit the second converted signal to the power supply device.

18. The medical device system according to claim 17, wherein the electrical circuit substrate further includes:
a detection circuit configured to detect whether the second connector is connected to the second receptacle, and
a switch circuit configured to stop supplying power from the power supply circuit to the medical device when the detection circuit detects that the second connector is not connected to the second receptacle.

19. The medical device system according to claim 15, wherein:
the connector of the medical device has a first shape, the first receptacle has a second shape, and the second receptacle has a third shape, and
the second shape prevents the connector of the medical device from being connected to the first receptacle and the third shape allows the connector of the medical device to be connected to the second receptacle.

20. A relay adapter, comprising:
a main body configured to be connected to a connector of a medical device; and
a first connector configured to be connected to a first receptacle of a power supply device configured to power the medical device, the first connector extending in an axial direction from a middle portion of the main body,
wherein:
the main body and the first connector form a T-shape, and the main body includes:
a lateral body extending in a direction intersecting the axial direction,
a second receptacle configured to be connected to the connector of the medical device, and
a grip provided at least one of (i) in an end surface of the lateral body in the second direction or (ii) between the second receptacle and the end surface.
